(19) United States
(12) Patent Application Publication
(10) Pub. No.: US 2007/0112371 A1
(43) Pub. Date: May 17, 2007

(54) Embolic protection filter having compact collapsed dimensions and method of making same

(75) Inventors: Vincent Cangialosi, Beverly, MA (US); Albert Dunfee, Byfield, MA (US); Terrence Dennis Belluche, North Hampton, NH (US); Joanne C. Gilbert, Amesbury, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/273,161

(22) Filed: Nov. 14, 2005

(51) Int. Cl. A61M 29/00

(52) U.S. Cl. 606/200

(57) ABSTRACT

An embolic protection filter for collecting particulate debris in a body lumen and method of making same. The embolic filter includes a spirally cut outer tubular member disposed about a spirally cut inner tubular member, the spiral cuts having opposite chirality. The embolic filter is transformable between a collapsed configuration and an expanded configuration wherein filter openings are defined by intersections of the spiral cuts. The spiral cuts may have a variable pitch for sizing the filter openings to be larger in a proximal portion of the filter for receiving embolic debris, and smaller in a distal portion of the filter for retaining embolic debris within the filter. In an embodiment of the invention, the length of the expanded configuration is substantially equal to the length of the collapsed configuration.

### FIELD OF THE INVENTION

The invention relates generally to intraluminal distal protection devices for capturing particulate in the vessels of a patient. More particularly, the invention relates to a filter for capturing emboli in a blood vessel during an interventional vascular procedure.

### BACKGROUND OF THE INVENTION

Catheters have long been used for the treatment of diseases of the cardiovascular system, such as treatment or removal of stenosis. For example, in a percutaneous transluminal coronary angioplasty (PTCA) procedure, a catheter is used to insert a balloon into a patient's cardiovascular system, position the balloon at a desired treatment location, inflate the balloon, and remove the balloon from the patient. Another example is the placement of a prosthetic stent in the body on a permanent or semi-permanent basis to support weakened or diseased vascular walls to avoid closure or rupture thereof.

These non-surgical interventional procedures often avoid the necessity of major surgical operations. However, one common problem associated with these procedures is the potential release of atherosclerotic or thrombotic debris into the bloodstream that can embolize distal vasculature and cause significant health problems to the patient. For example, during deployment of a stent, it is possible for the metal struts of the stent to cut into the stenosis and shear off pieces of plaque which become embolic debris that can travel downstream and lodge somewhere in the patient's vascular system. Further, particles of clot or plaque material can sometimes dislodge from the stenosis during a balloon angioplasty procedure and become released into the bloodstream.

Medical devices have been developed to attempt to deal with the problem created when debris or fragments enter the circulatory system during vessel treatment. One technique includes the placement of a filter or trap downstream from the treatment site to capture embolic debris before it reaches the smaller blood vessels downstream. A filter placed in the patient's vasculature before or during treatment of the vascular lesion can collect embolic debris in the bloodstream.

It is known to attach an expandable filter to a distal end of a guidewire or guidewire-like member that allows the filtering device to be placed in the patient's vasculature. The guidewire allows the physician to steer the filter to a location downstream from the area of treatment. Once the guidewire is in proper position in the vasculature, the embolic filter can be deployed to capture embolic debris. Some embolic filtering devices utilize a restraining sheath to maintain a self-expanding filter in a collapsed configuration. Once the restraining sheath is retracted by the physician withdrawing the proximal end of the sheath extending outside the patient's body, the expandable filter will attempt to transform itself into its fully expanded configuration. The restraining sheath can then be removed from the guidewire allowing the guidewire to be used by the physician to deliver interventional devices, such as a balloon angioplasty catheter or a stent delivery catheter, into the area of treatment. After the interventional procedure is completed, a recovery sheath can be delivered over the guidewire using over-the-wire techniques to collapse the expanded filter (with the trapped embolic debris) for removal from the patient's vasculature. Both the delivery sheath and recovery sheath should be relatively flexible to track over the guide wire and to avoid straightening the body vessel once in place.

Another distal protection device known in the art includes a filter mounted on a distal portion of a hollow guidewire or tube. A moveable core wire is used to open and close the filter. The filter is coupled at a proximal end to the tube and at a distal end to the core wire. With the physician manipulating a proximal portion of the device outside the patient's body, pulling on the core wire while pushing on the tube draws the ends of the filter toward each other, causing the filter framework between the ends to expand outward into contact with the vessel wall. Filter mesh material is mounted to the filter framework. To collapse the filter, the procedure is reversed, i.e., pulling the tube proximally while pushing the core wire distally to force the filter ends apart. A sheath catheter may be additionally used as a retrieval catheter at the end of the interventional procedure to reduce the profile of the “push-pull” filter, as due to the embolic particles collected, the filter may still be in a somewhat expanded state. The retrieval catheter may be used to further collapse the filter and/or smooth the profile thereof, so that the filter guidewire may pass through the treatment area without disturbing any stents or otherwise interfering with the treated vessel.

However, regardless of how a distal protection filter is expanded during a procedure, i.e., sheath delivered or by use of a push-pull mechanism, a crossing profile of the collapsed filter is to be at a minimum to reduce interference between the filter and other interventional devices or in-placed stents. As well, a compact filter profile is beneficial in crossing severely narrowed areas of vascular stenosis. Furthermore, it is advantageous for a filter to have a short or compact collapsed length, which allows the filter to be utilized in vessels that have minimal space distal to a stenosis. Thus, what is needed is a filter that achieves a reduced profile and/or a compact collapsed length without sacrificing the strength and stability needed for effective embolic capture and retention.

### BRIEF SUMMARY OF THE INVENTION

The present invention is a filter for collecting debris in a body lumen. The filter is constructed of an outer tubular member having a first spiral cut along a length thereof and an inner tubular member having a second spiral cut along a length thereof. The first spiral cut and the second spiral cut have opposite chirality, such that the first spiral cut is a right-handed helix and the second spiral cut is a left-handed helix. In an alternate embodiment, the first spiral cut may be a left-handed helix and the second spiral cut a right-handed helix without departing from the scope of the invention. The inner tubular member is coaxially positioned within the outer tubular member to situate the second spiral cut within the first spiral cut, such that when the filter is in an expanded configuration filter openings are defined by intersections between the first and second spiral cuts. The pitch of the first and second spiral cuts may be varied as the cuts extend distally. The pitch may also be decreased as the cuts extend distally to obtain filter openings in a proximal portion of the filter that are larger than filter openings in a distal portion of the filter. In another embodiment, the pitch of the first and second spiral cuts may be held constant over the proximal portions of the inner and outer tubular members and decreased over the distal portions of the inner and outer tubular members to obtain filter openings in a proximal portion of the filter that are larger than filter openings in a distal portion of the filter.

At least one of the inner or outer tubular members is preferably made from a metallic material, such as stainless steel, nitinol, or a cobalt-chromium super alloy. Accordingly, the filter may be heat treated in an expanded or a collapsed configuration to retain its shape. In another embodiment, the outer tubular member may be made from a polymeric material, such as polyethylene block amide copolymer, polyvinyl chloride, polyethylene, polyethylene terephthalate, polyamide, or polyimide. In another embodiment, each of the inner and outer tubular members is a metallic hypotube.

A method of making an embolic filter is also disclosed. The method includes cutting a left-handed helix in a section of outer tubing, cutting a right-handed helix in a section of inner tubing, and slidably inserting the inner tubing into the outer tubing, such that the right-handed helical cut is disposed within the left-handed helical cut. The steps of helically cutting the inner and outer tubing may include varying the pitch of the cuts, such as to gradually decrease the pitch of the cuts as they extend distally. In one embodiment, the method includes securing the distal end of the outer tubing to the distal end of the inner tubing and rotating the proximal end of the inner tube in one direction while rotating the proximal end of the outer tube in an opposite direction to expand the first and second helical cuts into the form of an expanded filter subassembly. The expanded filter subassembly is then heat treated in the expanded configuration to set the size and shape of the filter and the filter openings. In another embodiment, the inner and outer tube subassembly is longitudinally stretched to create elastically deformable open coils. A filter-shaped mandrel is then inserted between the coils to be positioned within the inner and outer tube subassembly. The coils are then arranged around the mandrel and heat treated to set the size and shape of the filter and the filter openings in the expanded filter configuration.

A method according to the present invention may also include selecting metallic hypodermic tubing for at least one of the inner and outer tubes, or for each of the inner and outer tubes. In another embodiment, polymeric tubing may be selected for the outer tube, and a shape memory alloy may be selected for the inner tube.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an illustration of a filter system in accordance with an embodiment of the present invention deployed within a blood vessel.

FIG. 2 is an illustration of a filter system in accordance with an embodiment of the present invention deployed within the coronary arterial anatomy.

FIG. 3 is a perspective view of a portion of a distal protection device in accordance with the present invention.

FIG. 3A is an enlarged cross-sectional view of the device of FIG. 3 taken along the line A-A.

FIGS. 4A-4E illustrate a method of making a filter in accordance with an embodiment of the present invention.

FIGS. 5A-5D illustrate another method of making a filter in accordance with an embodiment of the present invention.

FIG. 6 is a sectional view of a distal portion of a distal protection device in accordance with another embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

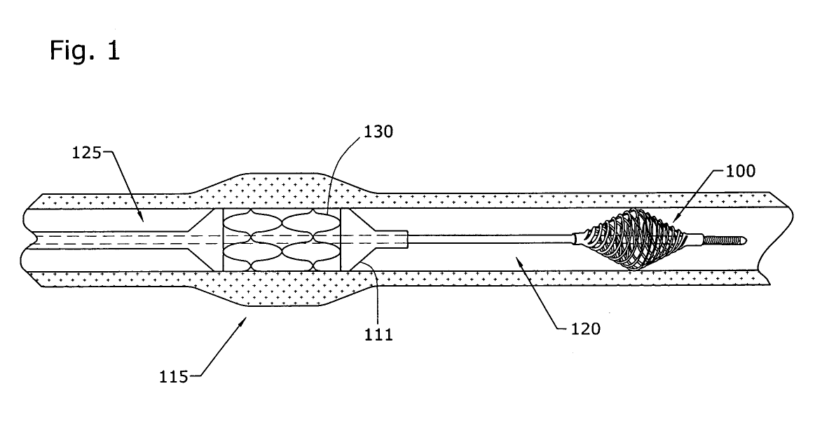

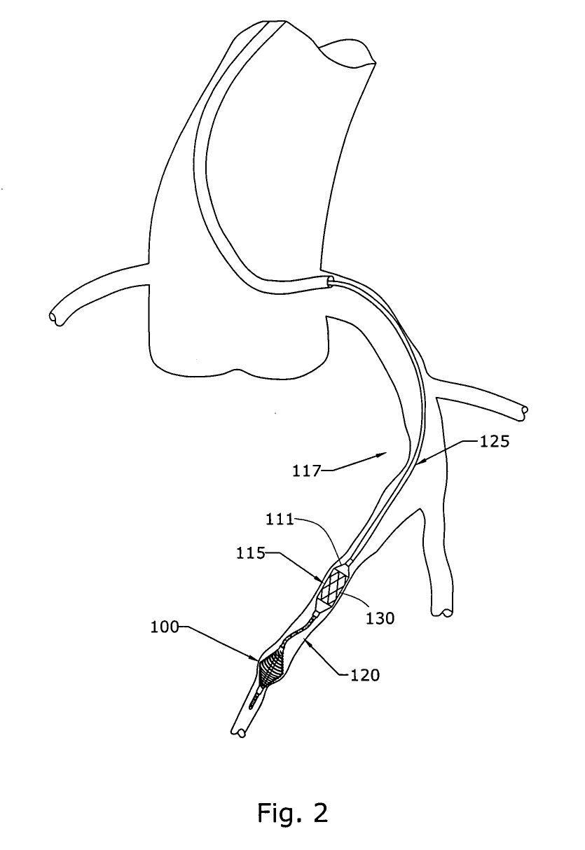

The present invention is a temporary distal protection device for use on a filter guidewire in minimally invasive procedures, such as vascular interventions or other procedures, where the practitioner desires to capture embolic material that may be dislodged during the procedure. With reference to FIGS. 1 and 2, deployment of balloon expandable stent 130 is accomplished by threading catheter 125 through the vascular system of the patient until stent 130 is located within a stenosis at predetermined treatment site 115. Once positioned, balloon 111 of catheter 125 is inflated to expand stent 130 against the vascular wall to maintain the opening. Stent deployment can be performed following treatments such as angioplasty, or during initial balloon dilation of the treatment site, which is referred to as primary stenting.

Catheter 125 is typically guided to treatment site 115 by a guidewire. In cases where the target stenosis is located in tortuous vessels that are remote from the vascular access point, such as with the coronary arteries 117 as shown in FIG. 2, a steerable guidewire is commonly used. According to an embodiment of the present invention, a filter guidewire generally designated as 120 guides catheter 125 to treatment site 115 and includes distally disposed filter 100 to collect embolic debris that may be generated during the procedure.

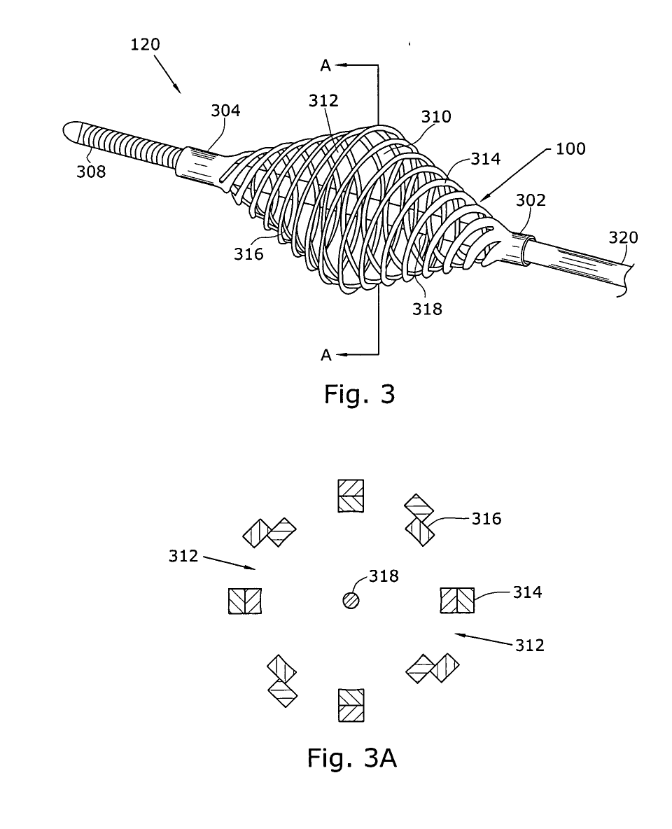

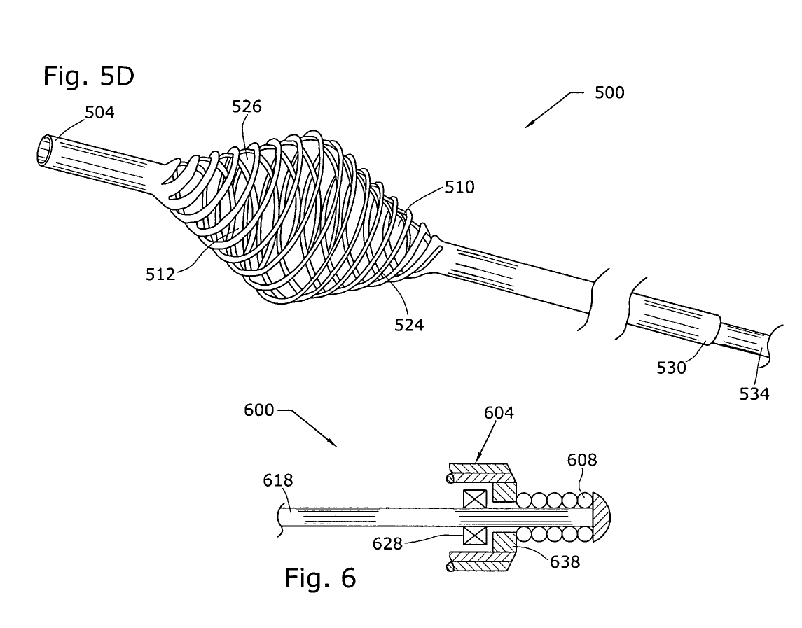

The present invention is directed to a distal protection device, viz., temporary embolic filter 100, which has a reduced profile in its collapsed configuration or state. In the embodiment shown in FIG. 3, filter guidewire 120 includes a hollow proximal shaft 320 in sliding relationship with core wire 318. Embolic filter 100 is axially secured at its proximal end 302 to a distal portion of proximal shaft 320 and at its distal end 304 to core wire 318. Filter ends 302, 304 may be spot welded, laser welded or secured using a bonding sleeve or adhesive to proximal shaft 320 or core wire 318, respectively, as would be apparent to one skilled in the relevant art. FIG. 3A is a sectional view of a distal portion of filter guidewire 120, taken along line A-A. FIG. 6 illustrates an alternate arrangement for joining filter distal end 304 to core wire 318. In this embodiment, filter distal end 604 is affixed to a cylindrical collar or bearing 638, such that core wire 618 may rotate relative to filter 600. Filter distal end 604 is held in its axial position relative to core wire 618, proximally by stop 628 and distally by flexible tip 608.

Core wire 318 may be made from a metal, such as nitinol, or a stainless steel wire. In an embodiment of the present invention (not shown), core wire 318 may be tapered at its distal end and/or be comprised of one or more core wire sections. Core wire 318 may be ground down and have several diameters in its profile in order to provide a stiffness transition. Core wire 318 has a proximal end (not shown) that extends outside of the patient from a proximal end (not shown) of proximal shaft 320. Core wire 318 also has coiled portion 308 with windings that extend from distal end 304 of filter 100. Coiled portion 308 may be a separate component from core wire 318, such as a flexible coil spring 608 shown in FIG. 6 that is formed from a round or flat coil of stainless steel and/or one of various radiopaque alloys such as platinum, as is well known to those of skill in the art of medical guidewires.

In another embodiment of the present invention, proximal shaft 320 may be constructed of multiple shaft components of varying flexibility to provide a gradual transition in flexibility as the shaft extends distally. Such a shaft arrangement is disclosed in U.S. Pat. No. 6,706,055, which is incorporated by reference herein in its entirety. In addition, a liner or axial bearings (not shown) as disclosed in the '055 patent may be utilized between core wire 318 and proximal shaft 320 in order to facilitate sliding movement there between during expansion and collapse of filter 100. In another embodiment, proximal shaft 320 may be a hollow tube enabling filtering device 120 to also function as a medical guidewire.

As illustrated in FIGS. 3 and 3A, embolic filter 100 includes a thin-walled, outer tubular member 314 surrounding a thin-walled, inner tubular member 316. When filter 100 is in its expanded configuration, filter openings 310, 312 are defined by the intersections of spiral cuts, as described below. Proximal filter openings 310 are larger than distal filter openings 312. Accordingly, proximal filter openings 310 are of a shape and size for receiving particulate debris there through, and distal filter openings 312 are sized for collecting embolic debris within filter 100 while permitting fluid to flow there through, such as blood flow sufficient for perfusion of body tissues. Optionally, radiopaque markers (not shown) may be placed on proximal and distal ends 302, 304 of filter 100 to aid in fluoroscopic observation during manipulation thereof. Filter 100 is sized and shaped such that when it is fully deployed, its greatest expanded diameter at approximately the midpoint of the filter will contact the inner surface of the blood vessel wall into which it is placed. The inner surface contact is preferably maintained over a substantial portion of the expanded circumference to prevent any emboli from escaping past filter 100.

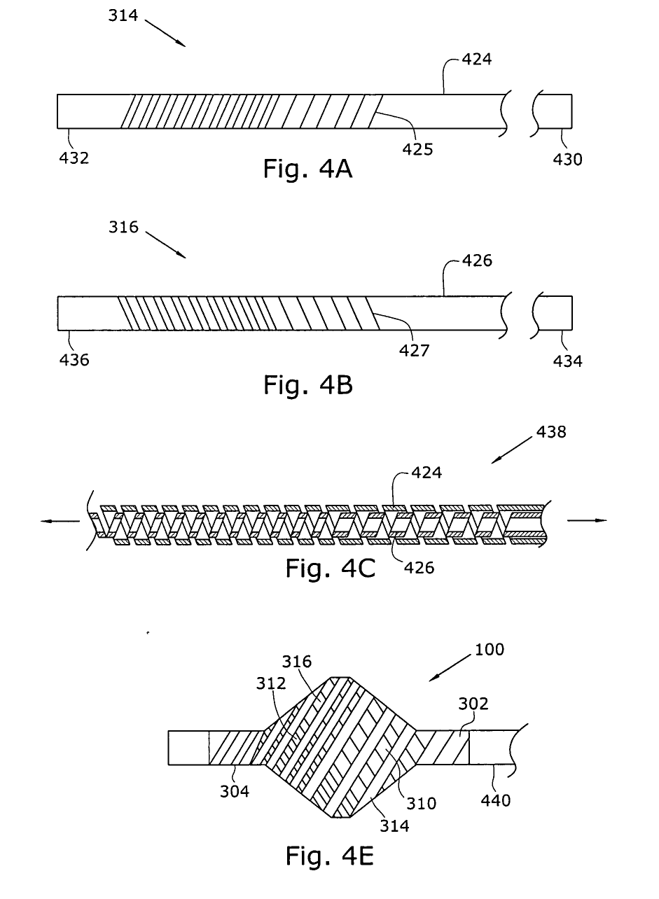

FIGS. 4A-4E illustrate a method of making filter 100 in accordance with an embodiment of the present invention. In FIG. 4A, a first, outer tube 424 having proximal end 430 and distal end 432 is selected of wall thickness and outer diameter to form expanded filter 100. A variable pitch spiral cut 425 is made in first tube 424, such that the pitch of the cut decreases as the cut extends from proximal end 430 to distal end 432. Spiral cut 425 may be a single, dual, or multi-helix. In this embodiment, a single, right-handed, spiral cut 425 is illustrated in FIG. 4A. The cut may be made by laser, water jet, electric discharge machine (EDM), or by any other suitable method known to one of skill in the art of making medical devices. Pitch is defined herein as the axial distance between the ends of one complete (360°) turn of a spiral cut. Spiral cuts, according to the invention, may be narrow slits that are spread open during elongation of the cut tube or expansion of the filter to form filter openings. Alternatively, spiral cutting may remove more material to form relatively broader slots. Coils or helical struts comprise the tube material that remains after spiral cuts, slits or slots are made are.

In FIG. 4B a second, inner tube 426 having proximal end 434 and distal end 436 is selected with an outer diameter that is slightly less than an inner diameter of first tube 424, such that an outer surface of second tube 426 will slide on an inner surface of first tube 424 during assembly. A variable pitch spiral cut 427 is made in second tube 426, such that the pitch of the cut decreases as the cut extends from proximal end 434 to distal end 436. Spiral cut 427 may be a single, dual, or multi-helix. In this embodiment, a single, left-handed spiral cut 427 is illustrated in FIG. 4B. It should be understood that inner tube cut 427 could be a right-handed spiral cut and outer tube cut 425 could be a left-handed spiral cut without departing from the scope of the present invention.

Second tube 426 is then slidably inserted within first tube 424, such that spiral cut 427 is situated within spiral cut 425 creating tubular subassembly 438, as shown in longitudinal cross-section in FIG. 4C. Tubular subassembly 438 is longitudinally stretched (see opposed force vector arrows) to open the spiral cuts thereby creating elastically deformable coils. A filter shaped mandrel, such as mandrel 440 shown in FIG. 4D, is then inserted between the coils to be positioned within the inner and outer tube subassembly 438. The coils are then arranged around mandrel 440 such that spiral cuts 425, 427 spread open and intersect with each other to form proximal and distal filter openings 310, 312 of filter 100, as shown in FIG. 4E. Filter openings 310, 312 may also be described as interstices between the coils or struts formed by spiral cuts 425, 427. A heat treatment is performed to set the size and shape of filter 100 and filter openings 310, 312 in the expanded filter configuration. Mandrel 440 is then removed by enlarging an opening 310 or 312 sufficiently to accommodate the removal without plastically deforming the filter. At any step after first and second tubes 424, 426 are nested together, they may be fixedly attached one to another at their proximal and distal ends by any suitable method known in the art of constructing medical devices.

In an embodiment of the present invention, both first and second tubes 424, 426 are comprised of a thin-walled, tubular structure of a metallic material, such as stainless steel, nitinol, or a cobalt-chromium super alloy. Such metallic tubing is commonly referred to as hypodermic tubing or a hypotube. Metallic tubing formed from other alloys, as disclosed in U.S. Pat. No. 6,168,571, which is incorporated by reference herein in its entirety, may also be used in the tubing of the present invention. When either of first or second tubes 424, 426 is made from a heat-treatable alloy, the filter subassembly is shaped into the configuration shown in FIG. 4E and, preferably, heat treated to set the filter shape, and particularly the various sizes of filter openings 310, 312 to form self-expanding filter 100.

In a second embodiment, second inner tube 426 is made from a metal alloy and first outer tube 424 is comprised of tubing made from a thermoplastic material such as polyethylene block amide copolymer, polyvinyl chloride, polyethylene, polyethylene terephthalate, polyamide, or a thermoset polymer such as polyimide. In this embodiment, inner tube 426 is arranged by itself around mandrel 440 in the configuration shown in FIG. 4E and is heat treated at a temperature suitable to set the filter shape in inner tube 426. If pre-shaped inner tube 426 alone has sufficient stiffness to provide structural support for filter 100, then mandrel 440 can be removed after the first heat treating step by sufficiently expanding one of the turns in spiral cut 427. Then, outer tube 424 is arranged about pre-shaped inner tube 426 to make tube subassembly 438. Finally, first and second tubes 424, 426 are fixedly attached one to another at their proximal and distal ends to create filter 100.

Optionally, if outer tube 424 in the second embodiment comprises a thermoplastic material, then subassembly 438 can be heat treated at a temperature suitable to set the filter shape in outer tube 424. The temperature for such a second heat treating step would be lower than the temperature of the first heat treating step due to the differences in thermal properties between themoplastics and metals. The second heat treating step can be performed with or without mandrel 440 supporting the assembly of tubes 424 and 426, depending on the radial strength provided by pre-shaped inner tube 426. If used, mandrel 440 is removed by enlarging an opening 310 or 312 sufficiently to accommodate removing the mandrel without plastically deforming the filter, as described in the previous example. The second embodiment can have a lower collapsed profile than the all-metal embodiment described above because plastic outer tube 424, i.e. polyimide, can be thinner than metal inner tube 426.

In accordance with the present invention, the pitch of spiral cuts 425, 427 may be varied at a constant or variable rate, depending on the desired final size of proximal and distal openings 310, 312. In one embodiment, the pitch may be held constant over the proximal portions of the length of first and second tubes 424, 426, and varied over the distal portions to achieve the desired opening sizes for a particular filter. Alternatively, the pitch may be held constant at a first pitch over the proximal portions of the length of first and second tubes 424, 426, and held constant at a second, smaller pitch over the distal portions.

In FIG. 3, filter 100 is shown in the deployed configuration. Filter 100 is transformable between its deployed, i.e., expanded, and collapsed configurations by relative movement between its ends. In the embodiment of FIG. 3, filter 100 is collapsed by distally advancing core wire 318 with respect to proximal shaft portion 320 to move filter distal end 304 away from filter proximal end 302. Filter 100 is returned to its deployed state by pulling core wire 318 proximally relative to hollow tube 320 to bring filter ends 302, 304 closer together, thereby allowing filter 100 to regain its expanded configuration. In further embodiments, a filter guidewire mechanism similar to that shown in any of the filter guidewires disclosed in U.S. Pat. Nos. 6,706,055, 6,818,006 and 6,866,677, which are incorporated by reference herein in their entireties, may be modified for use with filter 100.

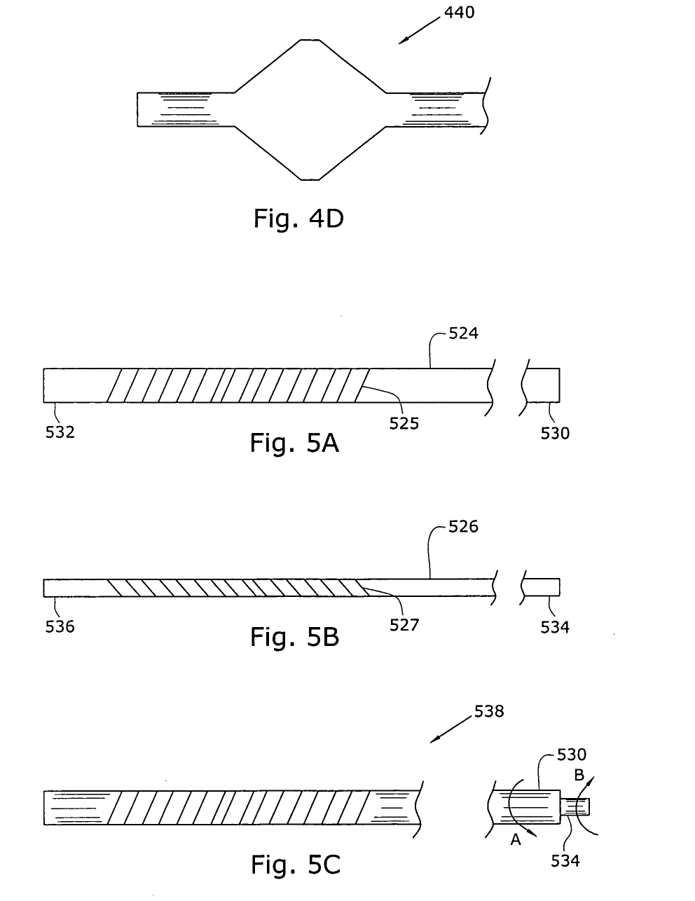

Alternatively, a filter in accordance with the present invention may be deployed and/or retrieved via a sheath catheter, such as by the method and apparatus disclosed in U.S. Pat. Nos. 6,059,814 and 6,346,116, which are incorporated by reference herein in their entireties. Further, the transformation of the filter may be impelled by external mechanical means alone or by self-shaping memory (either self-expanding or self-collapsing) within the filter materials. Preferably, filter 100 is self-expanding, meaning it has a mechanical memory to return to the expanded, or deployed configuration. As previously discussed, such mechanical memory can be imparted to the metallic tubing comprising filter 100 by thermal treatment to achieve a spring temper in stainless steel, for example, or to set a shape memory in a susceptible metal alloy, such as nitinol. Yet another method of transforming a filter in accordance with the invention between deployed and collapsed configurations is described infra with respect to FIG. 5D.

FIGS. 5A-5D illustrate a method of making filter 500 in accordance with another embodiment of the present invention. In FIG. 5A, a first, outer tube 524 having proximal end 530 and distal end 532 is selected with suitable dimensions to form expanded filter 500. A variable pitch spiral cut 525 is made in first tube 524. Spiral cut 525 may be a single, dual, or multi-helix. In this embodiment, a right-handed spiral cut 525 is illustrated in FIG. 5A. In FIG. 5B a second, inner tube 526 having proximal end 534 and distal end 536 is selected with an outer diameter that is slightly less than an inner diameter of first tube 524, such that an outer surface of second tube 526 will slide on or within an inner surface of first tube 524 during assembly. A variable pitch spiral cut 527 is made in second tube 526. Spiral cut 527 may be a single, dual, or multi-helix. In this embodiment, a left-handed spiral cut 527 is illustrated in FIG. 5B, such that spiral cuts 525, 527 have opposite chirality. As in the first embodiment, it should be understood that inner tube cut 527 could be a right-handed spiral cut and outer tube cut 525 could be a left-handed spiral cut without departing from the scope of the present invention.

Inner tube 526 is then inserted within outer tube 524, such that spiral cut 527 is situated within spiral cut 525, creating tubular subassembly 538, as represented in FIG. 5C. Filter distal end 504 is then formed by fixedly attaching first tube distal end 532 to second tube distal end 536 by any suitable method known in the art of constructing medical devices. The proximal ends 530, 534 are then rotated in opposite directions relative to one another, as indicated by counterclockwise and clockwise arrows A and B in FIG. 5C, to untwist or unwind first and second tubes 524, 526, thus radially expanding helical cuts 525, 527 and the coils or struts formed thereby into the expanded configuration of filter 500, as shown in FIG. 5D. In the expanded configuration of filter 500, filter openings 510, 512 are formed by the intersections of spread-open spiral cuts 525, 527. Filter openings 510, 512 may also be described as interstices between the coils or struts formed by spiral cuts 525, 527. If spiral cuts 525, 527 include varying pitch, as described above, then filter openings 512 can be larger than filter openings 510. Filter 500 thus formed is self-collapsing and relies on the relative rotation of proximal ends 530, 534 of first and second tubes 524, 526 to be expanded.

Optionally, a heat treatment may be performed on expanded tubular subassembly 538 to set a self-expanded size and shape of filter 500 and filter openings 510, 512. In the self-expanding embodiment, the proximal ends 530, 534 are rotated in opposite directions that are reversed relative to arrows A and B in FIG. 5C, to radially collapse filter 500 into the collapsed configuration. Both of the self-expanding and self-collapsing embodiments of filter 500 have an unexpanded, collapsed length that is substantially equal to the length of the expanded filter, allowing it to be used in procedures where insufficient space is available distal of the stenosis for positioning a filter having an elongated collapsed length.

FIG. 5D illustrates filter 500, without a core wire inserted, in its deployed configuration. Considered individually, either of spiral cut tubes 524 or 526 can be radially expanded or contracted by rotating one end with respect to the other. Tubes 524 and 526 require opposite end-to-end rotation for radial expansion because spiral cuts 525, 527 have opposite chirality, as described above. That is, a right-handed spiral can be expanded by rotating one end counterclockwise with respect to the other end, and a left-handed spiral can be expanded by rotating one end clockwise with respect to the other end. Alternatively, to contract tubes having oppositely chiral spirals, a right-handed spiral can be contracted by rotating one end clockwise with respect to the other end, and a left-handed spiral can be expanded by rotating one end counterclockwise with respect to the other end. Filter 500 is transformable between its deployed, i.e., expanded, and collapsed configurations by relative rotational movement between proximal ends 530, 534 of outer and inner tubes 524, 526. As such, filter 500 is collapsed by rotating outer tube proximal end 530 clockwise and inner tube proximal end 534 counterclockwise. Filter 500 is returned to its deployed state by reversing the rotation of proximal ends 530, 534, i.e., rotating outer tube proximal end 530 counterclockwise and inner tube proximal end 534 clockwise, thereby allowing filter 500 to regain its expanded configuration.

In an embodiment where outer and inner slotted tubes 524, 526 have unequal torsional stiffnesses, core wire 318 may be incorporated in the filter guidewire to provide rotational control of slotted tubes 524, 526. With core wire 318 affixed within filter distal end 504, outer tube proximal end 530 can be rotated with respect to core wire 318 to expand or collapse spiral cut 525. Similarly, inner tube proximal end can be rotated with respect to core wire 318 to expand or collapse spiral cut 527. Thus, by rotationally manipulating the proximal ends of core wire 318 and slotted tubes 524, 526 extending outside the patient, slotted tubes 524, 526 can be expanded or collapsed independently or simultaneously. For example, outer tube 524 can be rotationally expanded into the expanded configuration simultaneously or before inner tube 526 is expanded. Inner tube 526 can be rotationally collapsed simultaneously or before outer tube 524 is collapsed. For convenience, an accessory or tool (not shown) may be removably mounted to the proximal ends of core wire 318 and slotted tubes 524, 526 to aid in manually controlling and temporarily locking the relative rotational positions of the elements.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A filter transformable between a collapsed configuration and an expanded configuration for collecting debris in a body lumen, the filter comprising:
an outer tubular member having a first spiral cut along a length thereof that forms a first coiled portion; and
an inner tubular member having a second spiral cut along a length thereof that forms a second coiled portion, the first and second spiral cuts having opposite chirality, wherein the inner tubular member is coaxially disposed within the outer tubular member such that when the filter is in the expanded configuration filter openings are defined by intersections between the first and second coiled portions.

2. The filter of claim 1, wherein a pitch of at least the first spiral cut is varied along the cut.

3. The filter of claim 2, wherein the pitch of at least the first spiral cut decreases as the cut extends distally to provide filter openings in a proximal portion of the filter that are larger than filter openings in a distal portion of the filter.

4. The filter of claim 1, wherein at least one of the inner or outer tubular members is comprised of a metallic material selected from the group consisting essentially of stainless steel, nitinol, and cobalt-chromium super alloy.

5. The filter of claim 4, wherein the outer tubular member is comprised of a polymeric material selected from the group consisting essentially of polyethylene block amide copolymer, polyvinyl chloride, polyethylene, polyethylene terephthalate, polyamide, and polyimide.

6. The filter of claim 1, wherein each of the inner and outer tubular members is comprised of a material selected from the group consisting essentially of stainless steel, nitinol, and cobalt-chromium super alloy.

7. The filter of claim 6, wherein the inner and outer tubular members are comprised of the same material.

8. The filter of claim 1, wherein a pitch of the first and second spiral cuts is constant over a proximal portion of the filter and decreases over a distal portion of the filter to obtain filter openings in the proximal portion of the filter that are larger than filter openings in the distal portion of the filter.

9. The filter of claim 8, wherein the filter openings in the proximal portion of the filter are sized to receive embolic debris there through.

10. The filter of claim 1, wherein the first spiral cut is a left-handed helical cut and the second spiral cut is a right-handed helical cut.

11. The filter of claim 1, further comprising:
a filter distal end that includes a portion of the outer tubular member adjacent a distal end of the first spiral cut affixed to a portion of the inner tubular member adjacent a distal end of the second spiral cut; and
a filter proximal end that includes a portion of the outer tubular member adjacent a proximal end of the first spiral cut affixed to a portion of the inner tubular member adjacent a proximal end of the second spiral cut, wherein the filter is transformable between the collapsed configuration and the expanded configuration by axially translating the filter distal and proximal ends towards or away from each other.

12. The filter of claim 1, further comprising:
a filter distal end that is formed by affixing a portion of the outer tubular member adjacent a distal end of the first spiral cut to a portion of the inner tubular member adjacent a distal end of the second spiral cut, wherein the filter is transformable between the collapsed configuration and the expanded configuration by rotating a proximal end of the outer tubular member with respect to the filter distal end and rotating a proximal end of the inner tubular member with respect to the filter distal end, wherein the rotations of the inner and outer tubular members are in opposite directions.

13. The filter of claim 12, wherein a length of the expanded configuration of the filter is substantially equal to a length of the collapsed configuration of the filter.

14. The filter of claim 12, wherein a proximal portion of each of the inner and outer tubular members is long enough to extend out of the body lumen such that the proximal ends of the tubular members can be manually rotated by a clinician.

15. A filter guidewire comprising: the filter of claim 14; and an elongate core wire extending through the inner tubular member and being affixed within the filter distal end.

16. A method of making an embolic filter; comprising:
making a first spiral cut in a section of outer tubing;
making a second spiral cut in a section of inner tubing, the second spiral cut having opposite chirality to the first spiral cut;
inserting the inner tubing into the outer tubing so that the second spiral cut is disposed within the first spiral cut;
securing the outer tubing to the inner tubing at their respective ends to form a filter subassembly having a proximal end and a distal end;
longitudinally stretching the filter subassembly by its proximal and distal ends to open the first and second helical cuts thereby creating elastically deformable coils;
inserting a filter shaped mandrel between the coils to be positioned within the filter subassembly; and
heat treating the filter subassembly with the filter mandrel inserted therein to set the size and shape of the filter and filter openings in the expanded filter configuration.

17. The method of claim 16, further comprising: fitting the coils about the filter mandrel to form proximal filter openings that are larger than distal filter openings.

18. The method of claim 16, further comprising: selecting metallic hypodermic tubing for at least one of the inner and outer tubing.

19. The method of claim 16, wherein metallic hypodermic tubing is used for each of the inner and outer tubing.

20. The method of claim 16, wherein the steps of making the first and second spiral cuts in the inner and outer tubing includes varying the pitch of at least one of the first or second cuts.

21. The method of claim 16, wherein the first spiral cut is a right-handed helical cut and the second spiral cut is a left-handed helical cut.

22. The method of claim 16, further comprising: selecting a polymeric tubing for the outer tubing, wherein the inner tubing is of a metal alloy.

23. A method of making an embolic filter; comprising:
making a first spiral cut in a section of outer tubing having proximal and distal ends;
making a second spiral cut in a section of inner tubing, the second spiral cut having opposite chirality to the first spiral cut, the inner tubing having proximal and distal ends;
inserting the inner tubing into the outer tubing so that the second helical cut is disposed within the first helical cut;
securing the outer tubing to the inner tubing at their distal ends to form a filter subassembly;
rotating the proximal ends of the inner and outer tubings in opposite directions relative to one another to radially expand the spiral cuts; and
heat treating the filter subassembly with the spiral cuts radially expanded to set the size and shape of the filter and filter openings in the expanded filter configuration.

24. The method of claim 23, wherein the proximal end of the outer tubing is rotated in a clockwise direction and the proximal end of the inner tubing is rotated in a counterclockwise direction to radially expand the spiral cuts into the expanded configuration of the filter.

25. The method of claim 23, further comprising: selecting metallic hypodermic tubing for at least one of the inner and outer tubing.

26. The method of claim 23, wherein metallic hypodermic tubing is used for each of the inner and outer tubing.

27. The method of claim 23, wherein the steps of making the first and second spiral cuts in the inner and outer tubing includes varying the pitch of at least one of the first or second cuts.

28. The method of claim 23, wherein the first spiral cut is a right-handed helical cut and the second spiral cut is a left-handed helical cut.
